(19) United States
(12) Patent Application Publication
(10) Pub. No.: US 2003/0100873 A1
(43) Pub. Date: May 29, 2003

(54) Disposable pant diaper

(76) Inventors: Kent Hermansson, Vastra Frolunda (SE); Anna-Carin Elfstrom, Torslanda (SE); Karin Lindmark, Goteborg (SE); Linda Olsson, Goteborg (SE)

Correspondence Address: BURNS, DOANE, SWECKER & MATHIS, L.L.P., P.O. Box 1404, Alexandria, VA, 22313-1404, US

(21) Appl. No.: 10/284,190

(22) Filed: Oct. 31, 2002

Related U.S. Application Data
(60) Provisional application No. 60/330,778, filed on Oct. 31, 2001.

(51) Int. Cl. A61F 13/15

(52) U.S. Cl. 604/374; 604/378; 604/385.01

(57) ABSTRACT

A disposable absorbent pant diaper with a longitudinal direction and a transverse direction, an inner cover layer (11), an outer cover layer (12), and an absorption body (10) enclosed between the two cover layers (11, 12), and having two end edges (6, 7) extending in the transverse direction forming a waist opening and two side edges (8, 9) extending in the longitudinal direction forming leg openings, the pant diaper also having a front portion (1) and a rear portion (2) which are connected by an intermediate crotch portion (3). The absorption body (10) is constructed so that the liquid-spreading in the pant diaper is limited to remain below an area of spread of 200 square centimetres in the plane of the absorption body (10) for a liquid quantity of 150 millilitres.

### TECHNICAL FIELD

The present invention is directed to a disposable absorbent pant diaper with a longitudinal direction and a transverse direction, an inner cover layer, an outer cover layer, and an absorption body enclosed between the two cover layers, and having two end edges extending in the transverse direction forming a waist opening and two side edges extending in the longitudinal direction forming leg openings, the pant diaper also having a front portion and a rear portion which are connected by an intermediate crotch portion.

### BACKGROUND ART

A large number of users of absorbent pant diapers for different types of incontinence are physically active women who are not physically or mentally incapable of dealing with the exchange of said pant diaper.

The pant diapers for incontinent people which are available on the market today are aimed primarily at heavily incontinent people. These users are often physically disabled to a greater or lesser extent and, moreover, have often reached a mental state in which they are not aware of whether or when an evacuation of the bladder has taken place. The conventional type of pant diaper for heavy incontinence has proved to be extremely useful for this user group as there is a great need as far as this group is concerned for a product which can receive, spread and store large quantities of liquid at the same time as it makes close contact with the body so as to avoid leakage of discharged liquid. In order to achieve sufficient capacity for absorbing discharged liquid, and at the same time maintain the thinness of the article, the absorbent material included in a conventional pant diaper is spread out over a large area which, when the pant diaper is fitted on the wearer, extends from essentially the abdomen of the wearer via the crotch to the waist area on the back of the wearer. When a wearer stands or sits, discharged urine normally ends up in what is known as the wetting point which is located in the crotch portion of the diaper. As heavy incontinence often gives rise to several evacuations of the bladder, it is of vital importance that the pant diaper be designed so that liquid is effectively spread away from the wetting point in the crotch portion so as to be stored in the entirety of the available absorption material and also in order that the liquid-receiving wetting point is prepared for the next evacuation.

For a heavily incontinent person, it is obviously necessary for the discharged liquid to be distributed over the entire absorption area in order to ensure that leakage does not take place in the crotch portion which is subject to heavy liquid-loading. Inconvenience, in the form of discomfort and irritation, which results from large quantities of liquid being stored in the extensive absorption material and thus lying closely against large skin areas is something which has to be accepted for the high level of leakproofness afforded by the design of such a pant diaper.

In addition, a pant diaper designed for heavy incontinence is usually intended for people who are incapable of changing a used product themselves, people who are thus obliged to depend on outside help for replacing a used pant diaper. In this situation, it is not uncommon for there to be time for several evacuations to be discharged before exchange can take place at all. The pressure on the crotch area, which is exposed first to discharged liquid, must therefore of necessity be relieved for storage of the liquid in the absorbent front and rear portions of the pant diaper.

A further problem is the close fit afforded by the pant diaper in its safeguarding against leakage. Contrary to the purpose of the pant diaper, this close contact becomes a burden as no possibility is afforded for ventilating the surrounding skin areas which make contact with the liquid-filled absorption material.

An obvious problem arises when physically active women who are both physically and mentally well capable of dealing with changing their own incontinence product and are also fully aware of the undesirable presence of liquid next to the body are directed towards this type of product. In existing conventional heavy incontinence pant diapers, where it is expected that the product will have absorbed great quantities of liquid after several evacuations, the liquid has also been distributed over a large area of spread covering the abdomen and the rear portion of the wearer. The result of this is that, even if the wearer changes the product within a relatively short period of time, the spreading has already caused wet contact against large parts of the skin, which still remains when a new pant diaper is put on. This dampness feels uncomfortable and unfresh to the aware wearer and, moreover, spreads the characteristic, unpleasant odour of the discharged liquid, which is embarrassing and irritating in an active social life.

Another problem commonly encountered in conventional absorbent pant diapers is that, in order to satisfy the absorption requirement, these require large quantities of absorption material. The consequence of this is, of course, that the pant diaper takes on more bulky proportions and often feels uncomfortable to move in and difficult to conceal under normal clothes. As the target group, active women, are extremely active socially, it is of the utmost importance that the incontinence product not be noticed through clothes and to the greatest extent possible feels like normal underwear.

The problem of the great quantities of material used in production also makes the pant diapers more expensive to manufacture, which also makes them more expensive for the user. As said user group changes the pant diaper as promptly as possible after an evacuation, a relatively great number of pant diapers are used. A less expensive product would, of course, make it possible for the wearer to change more frequently and discard the product as soon as it is wet.

As is clear from the above, a need exists for an improved absorbent pant diaper intended for physically active women. It is also desirable to be able to offer an absorbent pant diaper which has low production costs.

### DISCLOSURE OF INVENTION

In the present invention, a pant diaper of the type referred to in the introduction has been produced, which pant diaper essentially eliminates the problems associated with previously known pant diapers for the physically active female target group.

In this connection, an absorbent pant diaper made according to the invention is characterized mainly in that the absorption body is constructed so that the liquid-spreading in the pant diaper is limited to preferably remain below an area of spread of 200 square centimetres in the plane of the absorption body for a liquid quantity of 150 millilitres.

The inevitable liquid-spreading in the conventional pant diaper which follows an evacuation will be identified immediately by an aware physically active woman who feels great discomfort at the situation. Said wearer will therefore change the used pant diaper within an essentially short period of time in order to avoid the unpleasant sensation of moving in a wetted product. As far as the psychological situation is concerned, it has also been found that the wearer intends to change pant diaper as soon as possible when an evacuation of the bladder has taken place. In contrast to physically and/or mentally disabled people, said target group can deal with an immediate change after a relatively large evacuation as it is the most natural reflex. As it is desirable for both reasons of comfort and reasons of stability that the absorption body extends slightly up towards the abdomen portion and the rear portion, it is of utmost importance that discharged liquid is, in the space of time between changes, prevented from spreading to these regions.

An absorbent pant diaper usually has an absorption body constructed from three layers, the absorption body being arranged between an inner preferably liquid-permeable layer facing the wearer and an outer preferably liquid-impermeable layer facing away from the wearer. The absorption body comprises absorbent parts, for example cellulose fluff pulp with or without the addition of what are known as superabsorbents, which are polymers capable of absorbing several times more liquid than their own weight, and components for the purpose of spreading liquid in the absorption body.

In one embodiment of the present invention, the absorbent pant diaper has been designed with an absorption body comprising a central absorption layer and a safety layer. The central absorption layer suitably includes a mixture of absorbent cellulose fluff pulp and, superabsorbents, one component having been modified so as to have an acidifying effect in contact with discharged liquid from the wearer in order to produce an absorption body with limited liquid-spreading. In order to achieve one object set for the invention, the absorption layer preferably comprises a liquid-absorbing material with high retention but with low spreading capacity. In one embodiment, the safety layer can also be provided with corresponding superabsorbent material as a guarantee against any possible leakage from the absorption layer. Using absorption material with a high degree of acidity is advantageous from the point of view of hygiene, as it is known that a low pH reduces the occurrence of negative effects on the skin and also checks the activity of microorganisms.

An absorption body according to the invention therefore advantageously comprises a pH-regulating substance in the form of, for example, a partly neutralized superabsorbent material, which means that the pH in the absorbent pant diaper will preferably be, after wetting, in the range of 3.5-4.9. The composition of the absorption material and the interrelationship between the absorption layer and the safety layer in a multilayered structure have also been designed so that, when the absorption body absorbs liquid discharged from the wearer, the liquid-spreading will be limited to a smaller area of the absorption body.

Even ordinary types of superabsorbent material can also be included in the absorption body, that is, material in the form of fibres, particles, granules, film or the like, which has the capacity to absorb liquid corresponding to several times the weight of the superabsorbent material itself. The superabsorbent material binds the liquid and forms a liquid-containing gel. These superabsorbents are well known to the person skilled in the art and do not therefore need to be described in detail.

The absorption body can also comprise other absorption materials besides a partly neutralized superabsorbent material, for example cellulose pulp. It has been found to be advantageous to use a partly neutralized superabsorbent material as above in combination with cellulose fluff pulp with a pH of less than 7, and preferably less than 6 which gives a further improved effect. The use of the above-mentioned absorbent materials with low pH values can, according to the invention, limit the spreading of liquid in an absorbent product.

A suitable cellulose pulp can include chemithermomechanical cellulose pulp with a pH=2.5-8.5, preferably 2.5-6.5, and most preferably 2.5-5.5, or of chemical cellulose pulp with a pH=2.5-8.5, preferably 2.5-8.0, and most preferably 2.5-7.0.

A suitable partly neutralized superabsorbent material can include, for example, a cross-linked polyacrylate of the type sold under the name HYSORB C 7110 by BASF. Other types of superabsorbent material than those indicated above, which have corresponding properties, can also be used. A suitable proportion of superabsorbent material in the absorption layer is 30-70%, preferably 45-55%, and most preferably around 50%, with regard to the weight of the material of the absorption body. As the absorption body has a multilayered structure, a safety layer is suitably also arranged therein. The safety layer can then also comprise superabsorbents so that additional safety against leakage is provided. A suitable proportion of superabsorbent in the safety layer is in this case between 5 and 25%, preferably around 15%.

The degree of neutralization in the superabsorbent material should normally be lower than 45% and preferably lower than 35%. However, preferably, the degree of neutralization should suitably be higher than about 20% so as to achieve the best effect as described in patent specification SE 511 838.

Superabsorbents are usually available in the form of separate particles, but fibres, flakes, granules and film also exist. The particles can be of any desired shape, for example spherical, spiral, half-spiral, cubic, rod-shaped, polyhedral etc. Shapes which have a great maximum dimension/minimum dimension ratio, such as needles, flakes and fibres, are also possible for use herein. Alternatively, conglomerates of superabsorbent particles can be used. In an absorption body according to the invention, use is preferably made of superabsorbent particles of a size not smaller than around 100 μm and not larger than around 1000 μm, and use is even more preferably made of particles where around 60-65% by weight of the particles are within a size range of around 106-500 μm and 40-35% by weight are in a size range of around 500-850 μm. The particle size has been established by the superabsorbent particles being sieved through sieves, where the mesh apertures of the sieves have previously known diameters. Those particles which passed through a sieve with a certain aperture size but not through a sieve with a smaller aperture size therefore have a size lying in a range between the two respective diameters.

The particle size is considered to be a significant factor for the rate at which superabsorbents absorb and spread liquid which has been absorbed. The liquid absorption rate for larger particles is considered to be slower on account of their limited liquid absorption surface area in relation to their volume. For a similar reason, extremely small particles are considered to have a higher liquid absorption rate but are in turn limited by the fact that, after swelling, they are easily forced into interfibre capillaries in a fibrous structure such as cellulose fluff layer. In addition, they also easily form a mass of coagulated gel which is held together by surface tension forces of the liquid, thus forming a liquid barrier, which in turn results in reduced liquid-spreading, a phenomenon usually known as gel blocking. The range of particle sizes mentioned above has been found to be favourable for achieving satisfactory absorption at the same time as the spreading of the absorbed liquid in the longitudinal direction is limited.

An absorbent pant diaper with limited liquid-spreading according to the invention means that the guaranteed fit of the pant diaper with its high level of leakproofness, a consequence of the close contact of the pant diaper with the body, benefits the wearer without moisture being confined where it can have negative repercussions on exposed skin areas. In order to guarantee the fit, the pant diaper can be provided with a number of elastic elements, for example stretchable threads, bands, film or the like. Using a large number of elastic elements prevents the elastic tightening feeling uncomfortable for the wearer by virtue of the fact that the elastic elements will individually not exert too great a local pressure on the wearer at the same time as the close contact of the pant diaper is guaranteed.

In order to benefit from the close contact with the body without moisture being caught in too extensive of an area in the space between the skin and the pant diaper, it is preferred that the absorption material has a limited extent inside the pant diaper. In a multilayered structure, the liquid-retaining absorption layer therefore has an extent in the longitudinal direction inside the pant diaper which should preferably not constitute more than 60-80%, and preferably around 65-70%, of the total extent of the absorption body. The liquid absorbed in the absorption layer thus makes contact with the skin of the wearer only via limited areas. For this reason, the total area of the absorption layer should preferably not exceed an area of 195-235 cm², and preferably of 210-220 cm², for liquid absorption of around 150 ml. The absorption body as a whole nevertheless has such an extent inside the pant diaper that the comfort and stability requirement is still met.

As mentioned previously, a reduced quantity of absorption material is desirable for reasons of convenience and comfort. The use of a smaller quantity of absorption material additionally means a significant environmental gain. Moreover, as users in the intended target group of the invention are assumed to change the product after a first evacuation of the bladder, it is also not necessary to have a larger quantity of absorption material than is expected to be needed in order to absorb all the liquid discharged on one evacuation. A pant diaper according to a first embodiment of the invention can therefore be constructed with a total absorption capacity of 350-400 g, preferably at most around 370 g. According to a second embodiment of the invention, in which the safety layer does not have the function of absorbing any liquid possibly leaking from the absorption layer, but its primary function is to guide liquid down into the absorption layer, the total absorption capacity can be slightly lower. Total absorption capacity of an absorption product means a theoretical value which is calculated with knowledge of the absorption capacity of the various component materials.

According to a first embodiment of the invention, the safety layer is positioned below the absorption layer, that is, the absorption layer lies next to the inner cover layer. The absorption body can be designed with a liquid-receiving layer next to the inner cover layer, and also an additional layer which constitutes a part of the absorption layer. In this embodiment, the safety layer can, as mentioned above, act as a safety barrier against leakage of liquid coming from the absorption layer. For this reason, the safety layer in this case suitably comprises a smaller proportion of superabsorbents, preferably 10-20% by weight, and most preferably around 15% by weight, of the total absorption material, for the purpose of binding liquid. Here, the safety layer has a lower density than the absorption layer, which can convey a softer impression to the pant diaper during use and thus increase the feeling of comfort.

In a second embodiment of the invention, the safety layer is positioned above the absorption layer, that is, the safety layer lies next to the wearer. In this case, the safety layer functions primarily as a liquid-transferring distance layer. In this embodiment, said layer has high porosity and is to a greater or lesser extent hydrophobic and thus has a good capacity for receiving liquid and discharging liquid to the absorption layer. In both the embodiments indicated, however, the safety layer can advantageously have a lower hydrophilicity than the absorption layer. In this embodiment, the composition of the safety layer results in the rapidly discharged liquid being transferred in a quick and concentrated manner directly into the absorption layer. As the liquid is transported straight into the absorption layer, perpendicularly to the plane of the absorption layer, initial spreading is counteracted. The liquid is thus accumulated in a more concentrated manner in the absorption layer and is thus, at an early stage, spread to a lesser extent in the longitudinal direction. The safety layer also constitutes a distance layer between liquid which has been absorbed in the absorption layer and the body of the wearer, and in this way prevents rewetting, that is, bodily fluid already absorbed escaping back through the inner cover layer. In addition, the safety layer serves as protection against spray when liquid is discharged and, by virtue of its extent, as an improvement in the comfort.

In both the above embodiments, and most advantageously in the latter case, it may be suitable to arrange a liquid-impermeable barrier layer below the absorption body next to the outer cover layer. This would afford increased protection against leakage.

In order to establish that a pant diaper according to the invention has the desired limitation of the spreading of discharged liquid, a pant diaper according to the invention was compared with a pant diaper comprising a conventional absorption body.

The products concerned were investigated by means of the user-like method, which method is designed so that the anatomy of the wearer and an evacuation of the bladder in relation to an absorbent product are imitated to the greatest possible extent. To this end, use was made of a measuring device constructed essentially according to the anatomy of a wearer.

### BRIEF DESCRIPTION OF DRAWINGS

The invention is described in greater detail below with reference to the figures shown in the accompanying drawings, in which:

FIG. 1 shows a pant diaper according to the invention, which pant diaper is in an unassembled state,

FIG. 2a shows a section along the line IIa-IIa through the pant diaper in FIG. 1 according to a first embodiment of the invention,

FIG. 2b shows a section along the line IIb-IIb through the pant diaper in FIG. 1 according to a second embodiment of the invention,

FIG. 3 shows a pant diaper according to the invention, which pant diaper is in the assembled state,

FIG. 4a shows a test fixture seen from the side, and

FIG. 4b shows the test fixture in FIG. 4a seen from above.

### MODE(S) FOR CARRYING OUT THE INVENTION

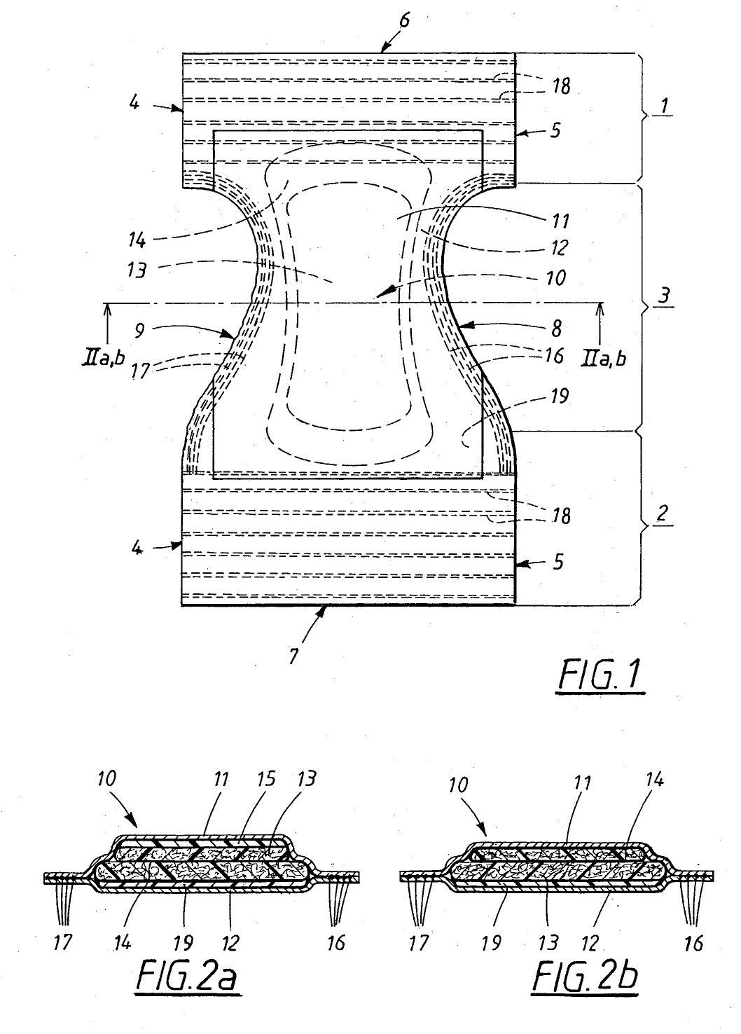

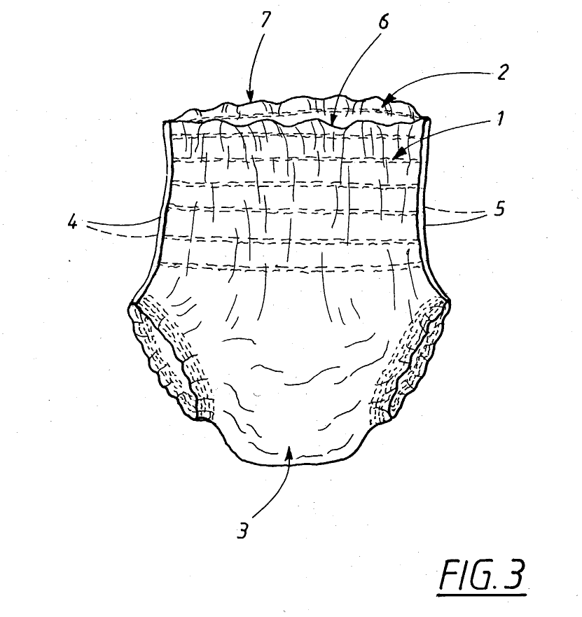

FIG. 1 shows an absorbent pant diaper in its as yet unassembled state, that is to say the waist and leg openings of the pant diaper have not yet been formed. The pant diaper comprises a front portion 1 intended to be fitted at the front on a wearer, a rear portion 2 intended to be fitted at the rear on the wearer, and a crotch portion 3 between the front and rear portions 1, 2, intended to be fitted between the legs of the wearer. The front portion 1 has two side edges 4, 5 for fastening together, which correspond to two side edges 4, 5 for fastening together on the rear portion 2, the respective edges 4, 5 being intended to be fastened together in order to form a pant diaper according to FIG. 3. The front and rear portions 1, 2 also have two straight end edges 6, 7 intended to form a waist opening. The crotch portion 3 has two longitudinal, inwardly curved, side edges 8, 9 intended to form leg openings. The pant diaper also comprises an absorption body 10 which is elongate in the longitudinal direction of the pant diaper and is enclosed between an inner cover layer 11, intended to face the wearer during use, and an outer cover layer 12, intended to face away from the wearer during use.

The front and rear portions 1, 2 and the crotch portion 3 of the pant diaper are made in one continuous piece which thus forms the outer cover layer 12. This outer cover layer 12 can include one or more layers of non-woven material or other textile-like materials.

The inner cover layer 11, which is located on that side of the absorption body 10 directed towards the wearer during use, has an hourglass-shaped design. The outer cover layer 12 is shaped so that it forms the entire extent of the pant diaper. The cover layers 11, 12 are interconnected around the absorption body 10 by, for example, gluing, stitching, or welding using heat or ultrasound. Furthermore, one or both of the cover layer(s) 11, 12 can be connected to the absorption body 10, by, for example, gluing, needling, stitching, or by welding using heat or ultrasound.

The inner cover layer 11 is of conventional type and can therefore include any liquid-permeable material suitable for the purpose. Examples of such materials are various types of thin non-woven material, perforated plastic films, net material, liquid-permeable foamed material or the like.

It is not necessary for the invention that the inner cover layer 11 include a separate material layer, but the cover layer 11 can be a surface of the absorption body 10 of the pant diaper. In this case, the two cover layers are not interconnected as described above.

The outer cover layer 12 can include the same type of material as the inner cover layer 11. As a measure to increase protection against leakage, a liquid-impermeable barrier layer 19 may be arranged between the absorption body and the outer cover layer 12, as shown in FIG. 2b. It may be an advantage if the barrier layer 19 has a certain breathability, that is, allows water vapour to pass through the layer 19. The liquid-impermeable barrier layer 19 can, like the inner cover layer 11, be an integral part of the absorption body and can include, for example, a liquid-impermeable skin-like surface on an absorbent foamed material. The barrier layer 19 is connected to the pant diaper by, for example, gluing, stitching, or welding using heat or ultrasound. Alternatively, the outer cover layer 12 may entirely or partly comprise a liquid-impermeable material, such as a liquid-impermeable plastic film, a non-woven layer which has been coated with a liquid-blocking material, or some other flexible material layer which has the capacity to resist liquid penetration.

In accordance with one embodiment of the invention, the absorption body 10 comprises two layers, on the one hand an absorption layer 13, and on the other hand a safety layer 14. Each of the two layers comprise a mixture, specific to each layer, of absorption material including cellulose fluff pulp and polymeric hydrocolloid materials, what are known as superabsorbents. Other suitable absorbent materials for use in the absorption body 10 are, for example, absorbent bound fibre layers, tissue layers, absorbent foam, peat or the like. Superabsorbents are materials with a capacity to absorb many times their own weight by forming a liquid-containing gel.

Superabsorbents are usually available in the form of particles, flakes, fibres, granules or the like. In some cases, the superabsorbent material may be present on its own, or it may be present together with another absorbent material and be arranged as layers, or it may be in the form of a mixture with other materials, such as cellulose fibres, or synthetic fibres.

Limitation of the liquid-spreading can be achieved when the absorption material has been prepared so that the degree of acidity, the pH, in the pant diaper lies, in connection with wetting, in a pH range of 3.5-4.9. This range is brought about by virtue of the fact that the absorption material comprises partly neutralized superabsorbent material with a degree of neutralization preferably higher than around 20%, lower than 45% and preferably lower than around 35%. A suitable superabsorbent material can include superabsorbent particles such as HYSORB C 7110 sold by BASF. Other types of superabsorbent material which have corresponding properties can also be used. The superabsorbent particles are usually in the form of separate particles, the superabsorbent particles preferably being larger than around 100 μm, but smaller than around 1000 μm, and even more preferably around 60-65% by weight of the particles being in the size range 106-500 μm and 40-35% by weight being in the size range 500-850 μm. The particle size of superabsorbent particles has been found to be a significant factor for the spreading of liquid, and thus the size of the superabsorbent particles should be selected so that the desired spreading effect is obtained. In order to achieve liquid-spreading according to the invention, said particle size range has been found to produce good results, which can be seen from the measurement values below.

In addition, an acidified cellulose pulp can also function as a pH-regulating material. The cellulose pulp can then include, for example, chemithermomechanical cellulose pulp with a pH=2.5-8.5, preferably 2.5-6.5, and most preferably 2.5-5.5, or of chemical cellulose pulp with a pH=2.5-8.5, preferably 2.5-8.0, and most preferably 2.5-7.0. A combination of a partly neutralized superabsorbent material as above with cellulose pulp with a pH of less than 7, preferably less than 6, has been found to give an improved effect. In order to obtain a suitable degree of acidity of the cellulose pulp, the pH of the latter can be controlled during the production process by, for example, addition of an acidifying substance. This added ingredient may be, for example, SO₂ water. By virtue of this procedure, the content of NaOH in the pulp can also be neutralized. Another way of obtaining a suitable degree of acidity of the cellulose pulp is to add a suitable acid to the finished pulp.

The desired limitation of liquid-spreading is not restricted to only the example mentioned above but can additionally be brought about in a number of ways, for example by using different layer compositions in the absorption body, differentiated compression zones in the absorption layer, loosely compressed pulp or the like. Alternatively, various liquid obstacles in the form of increased concentrations of superabsorbent material or barriers in the form of liquid-impermeable areas or the like can be arranged in parts of the absorption body.

The proportion of superabsorbent material in the absorption layer 13 is around 30-70%, preferably 45-55%, and most preferably around 50% of the weight of the material of the absorption layer, in order to achieve a satisfactory effect.

An absorption layer 13 and a safety layer 14 can have mutually different positions as far as close proximity to the body of the wearer is concerned. Each layer can thus be arranged next to either the inner cover layer 11 or the outer cover layer 12. The safety layer has a slightly greater extent in the longitudinal direction, and to some degree in the transverse direction, than the absorption layer 13. The positioning of the safety layer in each case is shown in the embodiments illustrated in FIGS. 2a and 2b. The safety layer 14 can serve a number of functions, some of which are increased leakproofness, guaranteed stability for the absorption body, and comfort for the wearer.

Elastic means 16, 17 are arranged along the side edges 8, 9 and form leg elastics when the pant diaper is used. Suitable elastic means are various types of elastic threads, bands, elastic non-woven fabric, elastic foamed material or the like. A number of transverse elastic elements 18, for example elastic threads, bands or the like, are fitted in a tensioned state transversely across both the front portion and the rear portion. The transverse elastic elements 18 are suitably enclosed between two layers of material, such as various types of non-woven fabric or other textile-like materials, which material constitutes the front and rear portions 1, 2 and the crotch portion 3 of the pant diaper.

FIGS. 2a and 2b show different embodiments of the absorption body in the pant diaper in FIG. 1. In FIG. 2a, the safety layer 14 is positioned below the absorption layer 13. In this embodiment, the absorption layer 13 is suitably designed with a liquid-receiving layer 15, which layer constitutes a part lying on top of the absorption layer, next to the inner cover layer 11. The liquid-receiving layer is characterized by high momentary liquid-absorbing capacity and the capacity to release liquid to the absorption layer 13. The liquid-receiving layer 15 advantageously has higher porosity than the absorption layer 13 and preferably includes resilient material layers. Moreover, it is in general advantageous if the layer has a lower hydrophilicity than the absorption layer 13 beyond it. The liquid-receiving layer 15 also constitutes a distance layer between liquid which has been absorbed in the absorption layer 13 and the body of the wearer and in this way prevents rewetting, that is to say bodily fluid already absorbed escaping back through the inner cover layer 11. Examples of materials which are usually used in the above-mentioned layer are polyester wadding, bulky non-woven materials, various types of laminate, cross-linked cellulose fibres or the like. A distance layer should additionally feel nice and soft against the body of the wearer. It is in general an advantage if the liquid-receiving layer 15 comprises a thermoplastic component which can be used for bonding the liquid-receiving layer 15 together with the inner cover layer 11 and/or the absorption layer 13.

The absorption layer 13 can also be provided with an additional layer which may be either a separate layer or constitute part of the absorption layer 13. An additional layer can have a liquid-transporting function, serve as a barrier layer against superabsorbent material comprised in the adjacent absorption layer or the like. An additional layer can include any materials suitable for the purpose and advantageously comprises a certain proportion of cellulose fluff pulp to the extent that it acts as a barrier layer.

In the illustrative embodiment shown in FIG. 2a, the pant diaper additionally comprises a safety layer 14 which is arranged next to the outer cover layer 12. The safety layer 14 has a greater extent in both the longitudinal direction and the transverse direction in relation to the absorption layer 13. The greater area gives a heightened sensation of comfort for the wearer and also stabilizes the absorption body in an extended position. In addition, the safety layer 14 serves as protection against any possible leakage from the absorption layer 13. In this embodiment, the layer 14 comprises the same type of material as the absorption layer, but with a different absorbent material composition. The superabsorbent proportion is slightly lower, between 10 and 20% and preferably around 15% of the total quantity of absorption material, the purpose of which superabsorbent particles being to absorb any liquid leaking from the absorption layer 13. The total theoretical absorption capacity of the whole absorption body is thus 350-400 g and preferably around 370 g. The safety layer 14 also has a lower density than the absorption layer 13, the lower density providing a more airy consistency, which in turn affords a softer feeling and thus increased comfort.

In FIG. 2b, the safety layer 14 is positioned above the absorption layer 13, lying in close proximity to the body of the wearer. In the figure, the safety layer 14 is shown with a slightly smaller transverse extent in the area of the section than the absorption layer 13. However, the two layers 13, 14 can advantageously have a similar transverse extent in said area. One advantage of the embodiment shown in FIG. 2b is that the safety layer 14, when positioned in this way, forms an effective liquid-admission layer for rapid and concentrated transport of liquid into the absorption layer 13. The discharged liquid is then collected in a central area, by means of which the subsequent spreading of liquid is already limited in an initial stage. In this embodiment, the safety layer 14 can, in the crotch portion 3 close to the wetting point, have a similar transverse extent to the absorption layer 13. A safety aspect is thus provided in that the safety layer, by virtue of its extent, can catch spray from a powerful evacuation of urine without the remaining material of the pant diaper being affected. In this embodiment, the safety layer 14 can serve a number of functions and can, for example, be a porous liquid-receiving layer, a liquid-transfer layer, a soft padding layer, a rewetting barrier, or a camouflage layer. As the safety layer 14 in this embodiment does not serve a partial function as an absorbent layer, the safety layer 14 does not comprise superabsorbent particles but includes materials characteristic of a liquid-receiving layer, suitably with higher porosity compared with the absorption layer 13 and with a good capacity for rapidly receiving liquid and for giving up liquid to the absorption layer 13. In this embodiment, the safety layer 14 can also constitute a distance layer between liquid which has been absorbed in the absorption layer 13 and the body of the wearer, by means of which rewetting is prevented. In order to provide a comfort-increasing function, it is an advantage if the safety layer 14 creates a pleasant and soft sensation. Suitable materials for use in the safety layer 14 may be polyester wadding, bulky non-woven materials, various types of laminate, cross-linked cellulose fibres or the like.

The absorption layer 13, located next to the outer cover layer 12, can have the same composition and quantity of absorption material as the first embodiment described in FIG. 2a. In the absence of an absorbent safety layer 14, the total theoretical absorption capacity is then reduced to 240-280 g. As a measure for safeguarding against leakage, a barrier layer 19 has in this case been arranged between the absorption layer 13 and the outer cover layer 12, which barrier layer suitably includes liquid-impermeable plastic film, a non-woven layer which has been coated with a liquid-blocking material or another material which resists liquid penetration. Alternatively, the outer cover layer 12 can be coated with a liquid-blocking material. A barrier layer is not always necessary as an ordinary evacuation should not exceed the stated absorption capacity of the pant diaper, especially as the product is aimed at women who are generally assumed to produce a smaller quantity of liquid. Another alternative, when a higher level of safety is desired, is for the reduced absorption capacity to be compensated by an increase in the quantity of absorption material in the absorption layer 13, so that a total absorption capacity of 350-400 g is again achieved. Barrier layers may also be an alternative here as a measure to increase protection against leakage.

FIG. 3 shows the pant diaper in its assembled form, seen in the direction towards the front portion 1, with the two longitudinal, inwardly curved side edges 8, 9 forming leg openings and the two end edges 6, 7 forming a waist opening. The two side edges 4, 5 for fastening together have in each case been joined together so that the pant diaper has a pant-like shape. The joining together can be effected by means of, for example, gluing, stitching, or welding using heat or ultrasound.

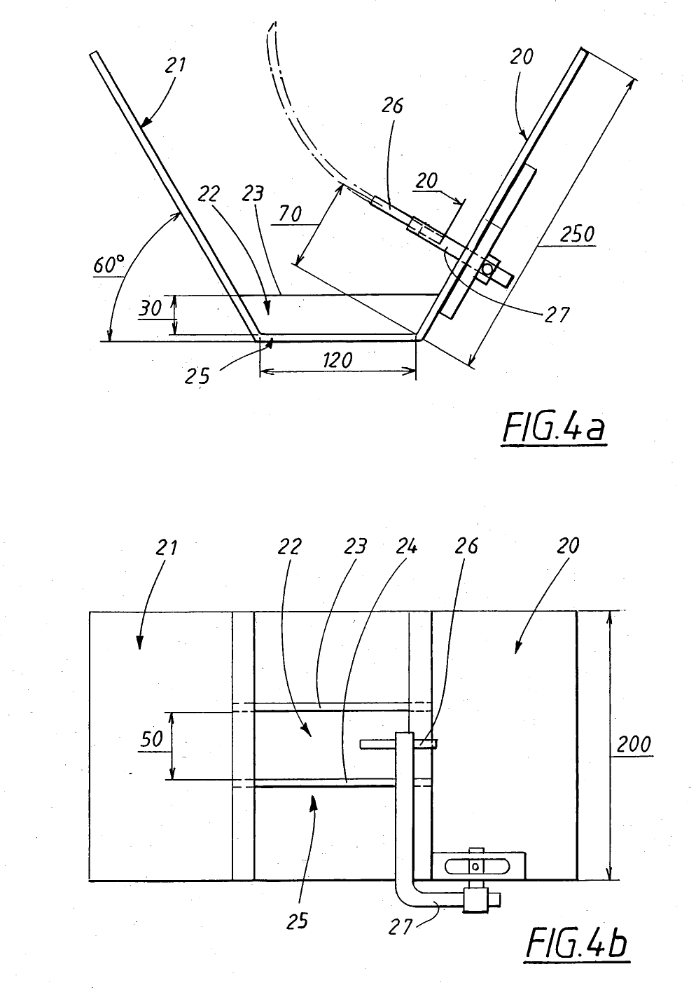

FIG. 4a represents the device, what is known as the fixture, on which test measurements were carried out, seen from the side. The fixture and the pump device were constructed so that the dimensions indicated in FIG. 4a, marked in millimetres, and the indicated angle of 60 (essentially correspond to the normal conditions existing between a wearer and the pant diaper fitted on the wearer. The fixture is constructed with a front surface 20, a rear surface 21 and a crotch area 22, on which surfaces the corresponding front portion 1, rear portion 2 and crotch portion 3 of the pant diaper can be arranged, that is to say inside the fixture. The crotch area 22 comprises two raised edges 23, 24 which rise above the bottom plate 25 of the fixture and that area of the bottom plate 25 delimited by said edges 23, 24. The raised edges 23, 24 are indicated in FIG. 4b, where the fixture is shown seen from above. When the crotch portion 3 of the pant diaper has been arranged on the crotch area 22, the crotch portion 3 will lie between the raised edges 23, 24 with the inner parts of the crotch portion lying against that part of the bottom plate 25 which is delimited by the raised edges 23, 24. A pump device is connected via a tube to a nozzle 26, from which synthetic urine is pumped out at a rate and in a volume corresponding to normal bladder evacuation. The nozzle 26 is fastened in the fixture via a holding arm 27 in a position perpendicular to the product when the latter has been arranged in the fixture. The holding arm 27 is secured in a side portion of the front surface 20 of the fixture and is arranged so that the arm is bent essentially perpendicularly across the front surface 20. As described above, the holding arm 27 holds the nozzle 26 in a fixed position above the absorbent product during the measurement procedure.

### DESCRIPTION OF EXAMPLE OF METHOD

The following example is intended to illustrate in greater detail the liquid-spreading effect in an absorbent pant diaper with an absorption body comprising a combination of a partly neutralized superabsorbent material and cellulose pulp with a pH of 2.5-8.5 compared with an absorption body comprising conventional materials of corresponding type.

Test Liquid

The test liquid was coloured synthetic urine, SUM. The composition of the synthetic urine was: 0.66 g/l MgSO₄, 4.47 g/l KCl, 7.60 g/l NaCl, 18.00 g/l NH₂CONH₂, 3.54 g/l KH₂PO₄, 0.745 g/l Na₂HPO₄, 0.4 g/l Nykockin (colour) and 1.00 g/l of a 0.1% solution of Triton X-100. The pH of this mixture was 6.0±0.5.

Method

For the measurements, an assembled pant diaper according to FIG. 3 was taken apart at the fastening side edges 4, 5 in order to obtain the shape of the pant diaper according to FIG. 1. The central point of the absorption body was measured and marked. Then a point, what is known as the wetting point, was measured seven centimetres from the central point in the direction towards the front portion 1. The pant diaper was positioned in a centred manner in the fixture so that its central point came to lie over a corresponding central point in the crotch area of the fixture. In this position, the wetting point marked on the absorption body came to lie at a distance of two centimetres directly below the nozzle. The synthetic urine was then pumped out at a rate of 20 ml/s with a dose volume of 150 ml, which volume is considered to correspond to a normal evacuation of liquid from the bladder. The pant diaper was left to rest in the fixture for five minutes so that the absorbed liquid was afforded an opportunity for spreading. After the stated time, the pant diaper was removed from the fixture, and the area of spread coloured by the test urine was marked at the contours. The pant diaper was placed in extended position on a plane surface, after which a transparent film of a specific area was fixed on top of said marked contour lines of the spreading. The contours were then drawn on the transparent film which was subsequently weighed in order to determine the weight per unit area of the film. The marked contours were then cut out, and the remaining part, corresponding to the liquid-spreading area, was weighed in order finally to be converted into area from the previously determined weight per unit area. Measurements were performed on on the one hand, ten pant diapers according to the present invention and, on the other hand, ten pant diapers comprising a conventional absorption body. The mean values with regard to weight measured in grams and liquid-spreading measured in square centimetres were then calculated for each type of pant diaper.

The measurement results showed clearly that a pant diaper according to the invention had markedly more limited liquid-spreading than a corresponding conventional pant diaper. A pant diaper according to the invention with a mean weight of 62.62 grams was found to have a mean area of spread of 192.98 cm² compared with the reference diaper with a weight of 55.06 grams and a mean area of spread of 240.01 cm². This gives a considerable difference of on average around 47 cm². In both series of measurements, there was a certain spread as far as the measurement values were concerned, which would probably correspond approximately to a normal discrepancy between individual pant diapers. On the whole, however, the liquid-spreading ranges of the two series of measurements did not overlap one another, and the highest measurement value of a pant diaper according to the invention was considerably lower than the lowest measurement value of a pant diaper of conventional type.

1. A disposable absorbent pant diaper with a longitudinal direction and a transverse direction, comprising: an inner cover layer, an outer cover layer, and an absorption body enclosed between the two cover layers, two end edges extending in the transverse direction forming a waist opening and two side edges extending in the longitudinal direction forming leg openings, and a front portion and a rear portion which are connected by an intermediate crotch portion, wherein the absorption body is constructed so that liquid-spreading in the pant diaper is limited to remain below an area of spread of 200 square centimetres in a plane of the absorption body for a liquid quantity of 150 millilitres.

2. A disposable absorbent pant diaper according to claim 1, wherein the absorption body comprises an absorption layer and a safety layer.

3. A disposable absorbent pant diaper according to claim 2, wherein the safety layer is positioned below the absorption layer.

4. A disposable absorbent pant diaper according to claim 2, wherein the safety layer is positioned above the absorption layer.

5. A disposable absorbent pant diaper according to claim 2, wherein the safety layer has a lower density than the absorption layer.

6. A disposable absorbent pant diaper according to claim 2, wherein the safety layer has a lower hydrophilicity than the absorption layer.

7. A disposable absorbent pant diaper according to claim 2, wherein the absorption layer has an extent in the longitudinal direction of the pant diaper which is smaller than an extent of the safety layer, corresponding to around 70%.

8. A disposable absorbent pant diaper according to claim 2, wherein a total area of the absorption layer does not exceed an area of 195-235 cm².

9. A disposable absorbent pant diaper according to claim 8, wherein the total area of the absorption layer does not exceed an area of 210-220 cm².

10. A disposable absorbent pant diaper according to claim 2, wherein the absorption layer comprises a liquid-absorbing material with high retention but with low spreading ability.

11. A disposable absorbent pant diaper according to claim 2, wherein a material of the absorption layer comprises chemithermomechanical cellulose pulp with a pH=2.5-8.5.

12. A disposable absorbent pant diaper according to claim 11, wherein the material of the absorption layer comprises chemithermomechanical cellulose pulp with a pH=2.5-6.5.

13. A disposable absorbent pant diaper according to claim 12, wherein the material of the absorbent layer comprises chemithermomechanical cellulose pulp with a pH=2.5-5.5.

14. A disposable absorbent pant diaper according to claim 2, wherein a material of the absorption layer comprises chemical cellulose pulp with a pH=2.5-8.5.

15. A disposable absorbent pant diaper according to claim 14, wherein the material of the absorption layer comprises chemical cellulose pulp with a pH=2.5-8.0.

16. A disposable absorbent pant diaper according to claim 15, wherein the material of the absorption layer comprises chemical cellulose pulp with a pH=2.5-7.0.

17. A disposable absorbent pant diaper according to claim 1, wherein the absorption body has a total absorption capacity of 350-400 g.

18. A disposable absorbent pant diaper according to claim 17, wherein the absorption body has a total absorption capacity of around 370 g.

19. A disposable absorbent pant diaper according to claim 10, wherein the liquid-absorbing material comprises superabsorbent material.

20. A disposable absorbent pant diaper according to claim 19, wherein the superabsorbent material constitutes 30-70%, with regard to a weight of the material of the absorption body.

21. A disposable absorbent pant diaper according to claim 20, wherein the superabsorbent material constitutes 45-55%, with regard to the weight of the material of the absorption body.

22. A disposable absorbent pant diaper according to claim 21, wherein the superabsorbent material constitutes around 50%, with regard to the weight of the material of the absorption body.

23. A disposable absorbent pant diaper according to claim 19, wherein the superabsorbent material includes particles of a size not smaller than around 100 μm and not larger than around 1000 μm.

24. A disposable absorbent pant diaper according to claim 23, wherein the superabsorbent material includes particles where around 60-65% by weight of the particles are within a size range of around 106-500 μm and 40-35% by weight are in a size range of around 500-850 μm.

25. A disposable absorbent pant diaper according to claim 19, wherein the superabsorbent material comprises neutralized superabsorbent particles with a degree of neutralization higher than around 20% but lower than 45%.

26. A disposable absorbent pant diaper according to claim 25, wherein the superabsorbent material comprises neutralized superabsorbent particles with a degree of neutralization lower than 35%.

27. A disposable absorbent pant diaper according to claim 1, wherein a liquid-impermeable barrier layer is arranged below the absorption body next to the outer cover layer.
